(19) United States
(12) Patent Application Publication
(10) Pub. No.: US 2021/0236468 A1
(43) Pub. Date: Aug. 5, 2021

(54) COMPOSITION FOR PREVENTING OR TREATING CANCER, COMPRISING A VASCULAR DISRUPTING AGENT AND TAXANE COMPOUND

(71) Applicant: Chong Kun Dang Pharmaceutical Corp., Seoul (KR)

(72) Inventor: Soo Jin Kim, Gyeonggi-do (KR)

(21) Appl. No.: 17/049,737

(22) PCT Filed: May 17, 2019
(86) PCT No.: PCT/KR2019/005941

§ 371 Date: Oct. 22, 2020

(30) Foreign Application Priority Data
May 18, 2018 (KR) 10-2018-0057131

(51) Int. Cl. A61K 31/426; A61K 9/00; A61K 31/337; A61P 35/00

(52) U.S. Cl. CPC A61K 31/426; A61K 9/0053; A61P 35/00; A61K 31/337; A61K 9/0029

(57) ABSTRACT

The present invention provides a composition for preventing or treating cancer, comprising (S)—N-(4-(3-(1H-1,2,4-triazole-1-yl)-4-(3,4,5-trimethoxybenzoyl)phenyl)thiazole-2-yl)-2-amino-3-methylbutanamide or pharmaceutically acceptable salts thereof and taxane compound or pharmaceutically acceptable salts thereof. The composition of the present invention shows an excellent effect of cancer treatment.

### TECHNICAL FIELD

The present invention relates to a composition for preventing or treating cancer, comprising a vascular disrupting agent (VDA) and taxane compound.

### BACKGROUND ART

A vascular disrupting agent (VDA) aims at selectively destroying the cytoskeletal microtubules of vascular endothelial cells and thus quickly and selectively disrupting tumor blood vessels formed therein, wherein the VDA may also induce ischaemic necrosis of cells located at the center of a tumor. Thus, a method for disrupting blood vessels by using the vascular disrupting agent (VDA) has recently emerged as a novel anti-cancer strategy. Accordingly, the present inventors have developed a compound of a following formula 1 as such vascular disrupting agent (the International Unexamined Patent Application Publication No. WO 2009-119980).

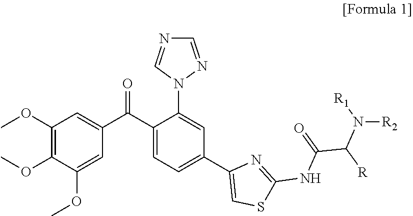

The compound of the formula 1 above is a tubulin polymerization inhibitor having a dual mechanism of action having: a fast collapse of pre-existing tumor blood vessels caused by destabilization of microtubules; and an apoptosis caused by a cell cycle arrest. However, in case of treating with the vascular disrupting agent (VDA) alone including the compound of the formula above, there is a problem in that a tumor may promptly regrow from a viable rim, thus reducing a therapeutic utility of such drugs.

Accordingly, the present inventors have attempted various researches to provide a composition capable of treating cancer as well as a method for treating cancer by making full use of an advantage of the vascular disrupting agent as an anti-cancer drug and solving a problem with a regrowth of the tumor.

### PRIOR ART REFERENCE

### Patent Document

WO2009/119980

### DISCLOSURE OF INVENTION

### Technical Problem

An objective of the present invention is to provide a composition for preventing or treating cancer, comprising a vascular disrupting agent and taxane compound.

Other objective of the present invention is to provide a method for treating cancer, comprising an administration of the vascular disrupting agent and the taxane compound into an individual in need.

Another objective of the present invention is to provide a use of the vascular disrupting agent and the taxane compound in the manufacture of a medicament for treating cancer.

### Solution to Problem

As a result of making a research effort to achieve the objectives above, the present inventors have completed a pharmaceutical composition for preventing or treating cancer, comprising a vascular disrupting agent (VDA) and taxane compound.

The vascular disrupting agent (VDA) aims at selectively destroying the cytoskeletal microtubules of vascular endothelial cells and thus quickly and selectively disrupting tumor blood vessels formed therein, wherein the VDA may also induce ischaemic necrosis of cells located at the center of a tumor.

In the present invention, the vascular disrupting agent is (S)—N-(4-(3-(1H-1,2,4-triazole-1-yl)-4-(3,4,5-trimethoxybenzoyl)phenyl)thiazole-2-yl)-2-amino-3-methylbutanamide represented by a following formula 2 or pharmaceutically acceptable salts thereof.

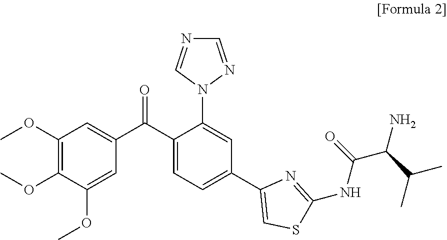 [SMILES: COc1cc(C(=O)c2ccc(-c3csc(NC(=O)[C@@H](N)C(C)C)n3)cc2-n2cncn2)cc(OC)c1OC]

In the present invention, a compound of the formula 2 above may be prepared, for example, by means of a method disclosed in the International Patent Publication WO 2009-119980, but not limited thereto.

In the present invention, the pharmaceutically acceptable salts of the compound of the formula 2 above mean salts conventionally used in a pharmaceutical industry. For example, there are inorganic ion salts prepared from calcium, potassium, sodium, magnesium or the like; inorganic acid salts prepared from hydrochloric acid, nitric acid, phosphoric acid, bromic acid, iodic acid, perchloric acid, sulfuric acid or the like; organic acid salts prepared from acetic acid, trifluoroacetic acid, citric acid, maleic acid, succinic acid, oxalic acid, benzoic acid, tartaric acid, fumaric acid, mandelic acid, propionic acid, lactic acid, glycolic acid, gluconic acid, galacturonic acid, glutamic acid, glutaric acid, glucuronic acid, aspartic acid, ascorbric acid, carbonic acid, vanillic acid or the like; sulfonic acid salts prepared from methanesulfonic acid, ethanesulfonic acid, benzenesulfonic acid, p-toluenesulfonic acid, naphthalenesulfonic acid or the like; amino acid salts prepared from glycine, arginine, lysine, etc.; amine salts prepared from trimethylamine, triethylamine, ammonia, pyridine, picoline, etc.; or the like, but types of salt meant in the present invention are not limited to those listed salts.

Particularly, a salt of (S)—N-(4-(3-(1H-1,2,4-triazole-1-yl)-4-(3,4,5-trimethoxybenzoyl)phenyl)thiazole-2-yl)-2-amino-3-methylbutanamide may be hydrochloride.

In the present invention, an active metabolite of the compound of the formula 2 above may be (4-(2-aminothiazole-4-yl)-2-(1H-1,2,4-triazole-1-yl)phenyl)(3,4,5-trimethoxyphenyl)methanone represented by a following formula 3. The term “active metabolite” is a substance, which actually shows a pharmacological activity within a subject to be treated, among substances occurring in a metabolic process of anabolism or catabolism in the body.

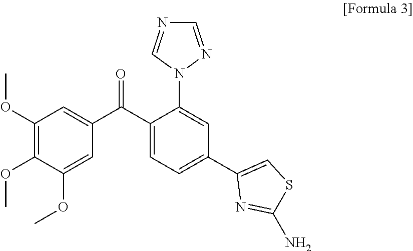 [SMILES: COc1cc(C(=O)c2ccc(-c3csc(N)n3)cc2-n2cncn2)cc(OC)c1OC]

In the present invention, the compound of the formula 2 contained in the pharmaceutical composition for preventing or treating cancer is present as the compound of the formula 3 above according to the metabolic process in an individual, and thus may show an effect of preventing, improving or treating cancer.

The compound of the formula 2 according to the present invention may quickly and selectively disrupt tumor blood vessels, and thus cause ischaemic necrosis of cells located at the center of a tumor. However, the vascular disrupting agent of the compound of the formula 2 above, etc. may allow the tumor to promptly regrow, thus reducing a therapeutic utility of drugs.

However, in the composition of the present invention, in case of co-administering (S)—N-(4-(3-(1H-1,2,4-triazole-1-yl)-4-(3,4,5-trimethoxybenzoyl)phenyl)thiazole-2-yl)-2-amino-3-methylbutanamide or pharmaceutically acceptable salts thereof with the taxane compound, an anti-cancer agent having a different therapeutic mechanism therefrom, it was identified that a cancer therapeutic activity thereof becomes very excellent thanks to a synergistic and complementary effect.

The taxane compound means a naturally occurring diterpene-based compound, which is a substance widely used in chemotherapy. The taxane compound has an effect of suspending a cell division by disturbing a production of microtubules, which deliver chromosomes to both poles during the cell division, and thus shows an effect of inhibiting a proliferation of cancer cells, which are divided more frequently than normal cells.

In the present invention, the taxane compound may be at least one selected from the group including paclitaxel, docetaxel, cabazitaxel, larotaxel, ortataxel and tesetaxel. Particularly, the taxane compound may be paclitaxel or docetaxel.

The pharmaceutically acceptable salts of the taxane compound mean salts conventionally used in a pharmaceutical industry. For example, there are inorganic ion salts prepared from calcium, potassium, sodium, magnesium or the like; inorganic acid salts prepared from hydrochloric acid, nitric acid, phosphoric acid, bromic acid, iodic acid, perchloric acid, sulfuric acid or the like; organic acid salts prepared from acetic acid, trifluoroacetic acid, citric acid, maleic acid, succinic acid, oxalic acid, benzoic acid, tartaric acid, fumaric acid, mandelic acid, propionic acid, lactic acid, glycolic acid, gluconic acid, galacturonic acid, glutamic acid, glutaric acid, glucuronic acid, aspartic acid, ascorbric acid, carbonic acid, vanillic acid or the like; sulfonic acid salts prepared from methanesulfonic acid, ethanesulfonic acid, benzenesulfonic acid, p-toluenesulfonic acid, naphthalenesulfonic acid or the like; amino acid salts prepared from glycine, arginine, lysine, etc.; amine salts prepared from trimethylamine, triethylamine, ammonia, pyridine, picoline, etc.; or the like, but types of salt meant in the present invention are not limited to those listed salts. Also, the taxane compound may be salt-free.

The composition of the present invention may be a composition containing (S)—N-(4-(3-(1H-1,2,4-triazole-1-yl)-4-(3,4,5-trimethoxybenzoyl)phenyl)thiazole-2-yl)-2-amino-3-methylbutanamide or pharmaceutically acceptable salts thereof and the taxane compound or pharmaceutically acceptable salts thereof; a combination containing (S)—N-(4-(3-(1H-1,2,4-triazole-1-yl)-4-(3,4,5-trimethoxybenzoyl)phenyl)thiazole-2-yl)-2-amino-3-methylbutanamide or pharmaceutically acceptable salts thereof and the taxane compound or pharmaceutically acceptable salts thereof; or a composition containing said combination. Thus, in the present invention, said composition may be used mixed with the combination.

The composition of the present invention may be valuably used for preventing or treating cancer. As the cancer, there are various cancers of the human body, gynecological tumor, endocrine system cancer, central nervous system tumor, ureteral cancer, etc. Particularly, the cancer includes lung cancer, gastric cancer, liver cancer, bone cancer, pancreatic cancer, skin cancer, head and neck cancer, skin melanoma, uterine cancer, ovarian cancer, rectal cancer, colorectal cancer, colon cancer, breast cancer, sarcoma of uterus, fallopian tube carcinoma, internal endometrium carcinoma, cervical carcinoma, vaginal carcinoma, vulvar carcinoma, esophagus cancer, laryngeal cancer, small bowel neoplasm, thyroid cancer, parathyroid cancer, sarcoma of soft tissue, urethral cancer, penis cancer, prostate cancer, multiple myeloma, chronic or acute leukemia, solid tumor of childhood, lymphoma (differentiated lymphoma and primary central nervous system lymphoma), bladder cancer, renal cancer, renal cell carcinoma, renal pelvic carcinoma, spinal axis tumor, brainstem glioma or pituitary gland adenoma. More particularly, the pharmaceutical composition of the present invention may be used for colorectal cancer, skin melanoma, lung cancer, gastric cancer, lymphoma or multiple myeloma, and still more particularly may be used for lung cancer.

The pharmaceutical composition of the present invention may be formulated into a preparation by using a pharmaceutically acceptable carrier according to a method easily practicable by those skilled in the technical field, to which the present invention pertains, such that such composition may be prepared in a unit dose form or prepared by being inserted into a multi-dose container.

The pharmaceutically acceptable carrier may be the one conventionally used in formulating a preparation, including, but not limited thereto, lactose, dextrose, sucrose, sorbitol, mannitol, starch, acacia rubber, calcium phosphate, alginate, gelatin, calcium silicate, microcrystalline cellulose, polyvinyl pyrrolidone, cellulose, water, syrup, methyl cellulose, methyl hydroxybenzoate, propylhydroxybenzoate, talc, magnesium stearate, mineral oil and the like. The pharmaceutical composition of the present invention may further contain a lubricant, humectant, sweetening agent, flavoring agent, emulsifier, suspending agent, preservative, etc. in addition to the components above. Suitable, pharmaceutically acceptable carriers and preparations are described in detail in Remington's Pharmaceutical Sciences (19th ed., 1995).

The composition of the present invention may contain two types of separate preparations, and may be also composed of one preparation.

In the composition of the present invention, (S)—N-(4-(3-(1H-1,2,4-triazole-1-yl)-4-(3,4,5-trimethoxybenzoyl)phenyl)thiazole-2-yl)-2-amino-3-methylbutanamide or pharmaceutically acceptable salts thereof and the taxane compound or pharmaceutically acceptable salts thereof may be administered simultaneously or at different times. After administering (S)—N-(4-(3-(1H-1,2,4-triazole-1-yl)-4-(3,4,5-trimethoxybenzoyl)phenyl)thiazole-2-yl)-2-amino-3-methylbutanamide or pharmaceutically acceptable salts thereof, the taxane compound or pharmaceutically acceptable salts thereof may be administered simultaneously or in one to five days later, particularly simultaneously or in one to three days later, and more particularly simultaneously or in one day later. The term “simultaneously” all includes:

administering the taxane compound or pharmaceutically acceptable salts thereof along with the administration of (S)—N-(4-(3-(1H-1,2,4-triazole-1-yl)-4-(3,4,5-trimethoxybenzoyl)phenyl)thiazole-2-yl)-2-amino-3-methylbutanamide or pharmaceutically acceptable salts thereof; and separately administering the taxane compound or pharmaceutically acceptable salts thereof no more than one day later, that is, on the same day after administering (S)—N-(4-(3-(1H-1,2,4-triazole-1-yl)-4-(3,4,5-trimethoxybenzoyl)phenyl)thiazole-2-yl)-2-amino-3-methylbutanamide or pharmaceutically acceptable salts thereof.

The composition of the present invention may be orally or parenterally administered (for example, applied intravenously, subcutaneously, intraperitoneally or locally) depending on a targeted method.

In the present invention, (S)—N-(4-(3-(1H-1,2,4-triazole-1-yl)-4-(3,4,5-trimethoxybenzoyl)phenyl)thiazole-2-yl)-2-amino-3-methylbutanamide or pharmaceutically acceptable salts thereof may be orally or parenterally administered.

In the present invention, the taxane compound or pharmaceutically acceptable salts thereof may be parenterally administered.

In the composition of the present invention, a suitable dosage of the effective components may vary in a range thereof depending on a patient's weight, age, gender, health condition, diet, administration time, administration method, excretion rate, severity of a disease, etc. A daily dosage of the inventive (S)—N-(4-(3-(1H-1,2,4-triazole-1-yl)-4-(3,4,5-trimethoxybenzoyl)phenyl)thiazole-2-yl)-2-amino-3-methylbutanamide or pharmaceutically acceptable salts thereof is about 1 mg/m 2 to 20 mg/m², preferably 5 mg/m 2 to 15 mg/m 2. Also, the daily dosage of the inventive taxane compound or pharmaceutically acceptable salts thereof may be the same or different depending on a type of the taxane compound. Particularly, the daily dosage of docetaxel is about 20 mg/m 2 to 150 mg/m², preferably 40 mg/m 2 to 80 mg/m 2. The daily dosage of paclitaxel is about 100 mg/m 2 to 300 mg/m², preferably 150 mg/m 2 to 250 mg/m 2.

Also, in the composition of the present invention, a suitable administration cycle of the effective components may be determined depending on the dosage. The inventive (S)—N-(4-(3-(1H-1,2,4-triazole-1-yl)-4-(3,4,5-trimethoxybenzoyl)phenyl)thiazole-2-yl)-2-amino-3-methylbutanamide or pharmaceutically acceptable salts thereof may be administered once a day to once every three weeks, particularly once a day to once a week, but not limited thereto.

Also, the taxane compound or pharmaceutically acceptable salts thereof may be administered once a day to once every three weeks, particularly once a week to once every three weeks.

The present invention provides a method for treating cancer, including an administration of (S)—N-(4-(3-(1H-1,2,4-triazole-1-yl)-4-(3,4,5-trimethoxybenzoyl)phenyl)thiazole-2-yl)-2-amino-3-methylbutanamide represented by the formula 2 or pharmaceutically acceptable salts thereof and the taxane compound or pharmaceutically acceptable salts thereof into an individual in need.

Particularly, provided is the method for treating cancer, including steps of: administering (S)—N-(4-(3-(1H-1,2,4-triazole-1-yl)-4-(3,4,5-trimethoxybenzoyl)phenyl)thiazole-2-yl)-2-amino-3-methylbutanamide represented by the formula 2 or pharmaceutically acceptable salts thereof into the individual in need; and administering the taxane compound or pharmaceutically acceptable salts thereof into the individual in need.

As used herein, the term “individual” includes a mammal, particularly a human. The therapeutic method includes an administration in a therapeutically effective amount, and the term “therapeutically effective amount” refers to an amount of the inventive (S)—N-(4-(3-(1H-1,2,4-triazole-1-yl)-4-(3,4,5-trimethoxybenzoyl)phenyl)thiazole-2-yl)-2-amino-3-methylbutanamide represented by the formula 2 or pharmaceutically acceptable salts thereof and the taxane compound or pharmaceutically acceptable salts thereof, which are effective in cancer treatment.

The present invention provides a use of (S)—N-(4-(3-(1H-1,2,4-triazole-1-yl)-4-(3,4,5-trimethoxybenzoyl)phenyl)thiazole-2-yl)-2-amino-3-methylbutanamide represented by the formula 2 or pharmaceutically acceptable salts thereof and the taxane compound or pharmaceutically acceptable salts thereof, in the manufacture of a medicament for treating cancer.

The composition containing the inventive (S)—N-(4-(3-(1H-1,2,4-triazole-1-yl)-4-(3,4,5-trimethoxybenzoyl)phenyl)thiazole-2-yl)-2-amino-3-methylbutanamide represented by the formula 2 or pharmaceutically acceptable salts thereof and the taxane compound or pharmaceutically acceptable salts thereof, which are used for preparing a drug, may be mixed with an acceptable carrier, etc., and further contain other additional agents.

Matters mentioned in the use, composition and therapeutic method of the present invention are equally applied, if not contradictory to each other.

### Advantageous Effects of Invention

A composition of the present invention shows a cancer-preventive or -therapeutic activity by containing a vascular disrupting agent and taxane compound, and has a remarkably excellent anti-cancer effect compared to a single effective component. Thus, the composition of the present invention may be applied for preventing, improving or treating cancer.

### BRIEF DESCRIPTION OF DRAWINGS

FIG. 1 is a graph of identifying that a tumor volume was remarkably decreased in a group dosed with CKD-516 and docetaxel together compared to a group dosed with CKD-516 or docetaxel alone as a result of comparing tumor volumes (H1975) thereof after the end of an experiment.

FIG. 2 is a graph of identifying that the group dosed with CKD-516 and docetaxel together shows not only an effect of inhibiting a growth of a cancer tissue but also an effect of decreasing the tumor volume as a result of comparing the tumor volumes (H1975) thereof during an experimental period.

FIG. 3 is a graph of identifying that a tumor volume was remarkably decreased in the group dosed with CKD-516 and docetaxel together compared to the group dosed with CKD-516 or docetaxel alone as a result of comparing the tumor volumes (A549) thereof after the end of the experiment.

FIG. 4 is a graph of identifying that the group dosed with CKD-516 and docetaxel together shows a remarkably excellent effect of inhibiting a growth of a cancer tissue as a result of comparing the tumor volumes (A549) thereof during the experimental period.

FIG. 5 is a graph of identifying that a tumor volume was remarkably decreased in the group dosed with CKD-516 and paclitaxel together compared to the group dosed with CKD-516 or paclitaxel alone as a result of comparing the tumor volumes (H460) thereof after the end of the experiment.

FIG. 6 is a graph of identifying that the group dosed with CKD-516 and paclitaxel together shows a remarkably excellent effect of inhibiting a growth of a cancer tissue as a result of comparing the tumor volumes (H460) thereof during the experimental period.

### MODE FOR THE INVENTION

Hereinafter, the configuration and effects of the present invention will be described in more detail through Examples. The following Examples are provided only for the purpose of illustrating the present invention, and thus the scope of the present invention is not limited thereto.

### <Example 1> Identification of a Synergy Effect Between the Compound of the Formula 2 and Docetaxel (1)

1. Experimental Method

Preparation for a Tumor Cell Line

As a human tumor cell line, a lung cancer cell line, i.e., H1975 cell lines were purchased from the American Type Culture Collections (ATCC). One vial of the cell lines was inserted into an RPMI1640 medium (Gibco, 22400) containing a heatinactivated 10% fetal bovine serum (FBS; Gibco, 10082-742), and cultured in a 5% CO₂ incubator at 37° C. The resulting cells were washed with PBS, after which a 10-fold dilution of 2.5% Trypsin-EDTA (Gibco, 15090) was added thereto, such that cells were isolated therefrom. Then, centrifugation was performed (at 1,000 rpm for 5 min), after which supernatant thereof was discarded, such that a cell suspension was obtained by means of a new medium. A survival rate was identified with a microscope to make a preparation in an amount of 1.0×10⁷ cells/mL.

Preparation for an Animal Model

The five-week old male thymic nude mice (Hsd: Athymic Nude-Foxn1^{nu}) were purchased from Saeron Bio Inc. (Gyeonggi Province in South Korea).

Thus prepared tumor cell lines were subcutaneously (sc) injected by 0.2 ml (2×10⁶ cells) into a back of the mice by using a 26-gauge needle syringe. In about three to four weeks after the injection, animals whose tumor sizes reached 170 mm³ to 220 mm³ were selected and used for an experiment.

Preparation for an Effective Component

The compound of the formula 2, i.e., CKD-516 was dissolved in 0.9% saline (excipient 1) and prepared in accordance with an administered dose for each group as shown in a following table 1.

Docetaxel was dissolved in an excipient (excipient 2), which was prepared by mixing cremophor, ethanol and saline at a ratio of 1:1:8, and prepared in accordance with the administered dose for each group as shown in the following table 1.

TABLE 1

| | | Amount of | |
|---|---|---|---|
| | Administered | administered | |
| Administered | dose | solution | Administration |
| substance | (mg/kg) | (ml/kg) | method |
| Negative control | — | 10 | ip |
| (excipient 2) | | | |
| Positive control | 6 | 10 | ip |
| (docetaxel) | | | |
| CKD-516 | 3 | 10 | ip |
| CKD-516 + | 3 + 6 | 10 + 10 | ip |
| Docetaxel | | | |

Drug Administration and Identification of Anti-Cancer Activity

An anti-cancer effect of CKD-516 and docetaxel was evaluated by using the prepared animal model. Experimental groups were randomly divided as shown in the table 1 above.
Excipient 2: Twice a week for three weeks, intraperitoneal administration;
Docetaxel: 6 mg/kg, twice a week for three weeks, intraperitoneal administration;
CKD-516: 3 mg/kg, twice a week for three weeks, intraperitoneal administration; and
CKD-516+Docetaxel: CKD-516 (3 mg/kg, twice a week for three weeks, intraperitoneal administration) to be administered first+Docetaxel (6 mg/kg, twice a week for three weeks, intraperitoneal administration) to be administered on the next day.

The anti-cancer activity was determined based on a tumor volume. The tumor volume was obtained by measuring a long axis and a short axis of the tumor by means of electronic callipers (CD-15CPX, Mitutoyo Corp., Japan) and calculating measured values according to a following equation (once a week):

Tumor volume (mm³)=(Long axis length×Short axis length²)/2.

Statistical Processing

A comparison between a positive control group and groups dosed with a test substance with regard to a negative control group, and a comparison between the positive control group and the groups dosed with a test substance were verified through One-way ANOVA. At this time, assuming that significance was accepted, a post-hoc test was performed with Duncan test, if an equal variance was accepted, and with Dunnett's test, if the equal variance was not accepted. If p-value is 0.05 or less, significance was accepted, and SPSS 10.1, a statistical program in common use, was used.

2. Experimental Results—Anti-Cancer Activity

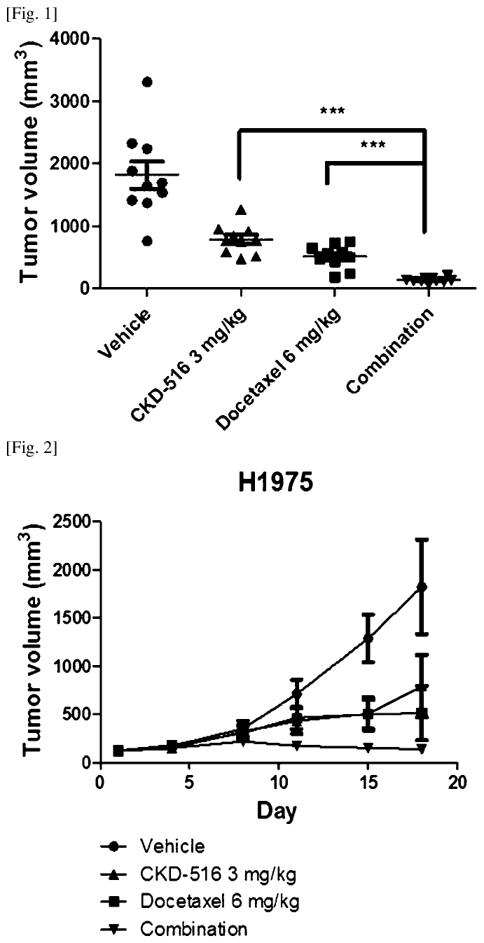

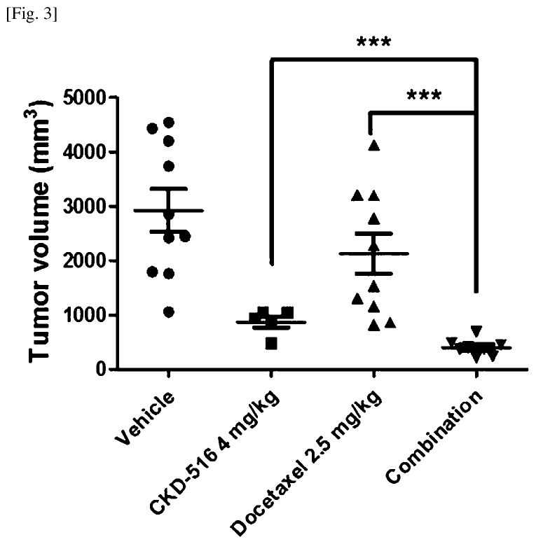

A tumor volume of the negative control group dosed with the excipient only was continuously increased over time. In case of the group dosed with docetaxel alone as a positive control substance, a growth of the tumor was decreased over time compared to the negative control group, but the tumor volume was continuously increased. Also, the group dosed with CKD-516 alone also showed a similar tendency to the group dosed with docetaxel. However, it was identified for the group dosed with CKD-516 and docetaxel together that the tumor volume is not significantly different from the initial tumor volume, but the tumor volume is remarkably small at the end of the experiment compared to the group dosed with docetaxel alone or CKD-516 alone (Table 2 and FIG. 1). Also, it was identified that the tumor volume, which used to be increased after the third administration, is decreased (Table 2 and FIG. 2).

It suggests that the co-administration of CKD-516 and docetaxel shows an effect of decreasing the tumor volume, which is not shown in the single administration, thanks to a synergy action between a blood vessel-destroying activity by CKD-516 and a cell division-suspending effect by docetaxel.

TABLE 2

| | | Tumor volume (mm³) | | | | | |
|---|---|---|---|---|---|---|---|
| Group | | 4/7 | 4/10 | 4/14 | 4/17 | 4/21 | 4/24 |
| Excipient | mean | 123.6 | 172.5 | 353.3 | 711.2 | 1286.0 | 1820.3 |
| | SD | 34.7 | 44.7 | 69.4 | 180.1 | 434.5 | 688.1 |
| Docetaxel | mean | 123.4 | 178.0 | 309.6 | 460.5 | 500.2*** | 513.9*** |
| | SD | 32.9 | 49.1 | 116.0 | 181.3 | 191.2 | 187.7 |
| CKD-516 | mean | 121.8 | 146.3 | 321.3 | 432.4* | 504.9 | 790.7*** |
| | SD | 31.0 | 49.7 | 167.2 | 173.4 | 149.8 | 233.5 |
| Co- | mean | 121.3 | 150.8 | 218.7 | 172.2*** | 150.0*** | 138.1*** |
| administration | SD | 29.2 | 41.0 | 94.5 | 49.1 | 44.6 | 38.6 |

### <Example 2> Identification of a Synergy Effect Between the Compound of the Formula 2 and Docetaxel (2)

1. Experimental Method

Preparation for an Animal Model

The four-week old male Balb/c nude mice were purchased from Central Lab Animal Inc.

A-549 cancer cell lines (1×10⁷ cells), which are human lung cancer cell lines purchased from the ATCC, were subcutaneously injected into the mice. Particularly, a needle point was inserted into mice in the left side before injecting cancer cells, and moved from side to side to inject the cancer cells and to identify that an administered solution was not leaked out, such that a tumorigenesis was observed. Animals whose tumor sizes reached 100 mm³ to 250 mm³ were selected and used for an experiment.

Preparation for an Effective Component

The compound of the formula 2, i.e., CKD-516 was dissolved in purified water and prepared in accordance with an administered dose for each group as shown in a following table 3.

Docetaxel was dissolved in an excipient (excipient 3), which was prepared by mixing ethanol, Tween 80 and saline at a ratio of 1:1:8, and prepared in accordance with the administered dose for each group as shown in the following table 3.

TABLE 3

| | | Amount of | |
|---|---|---|---|
| | Administered | administered | |
| Administered | dose | solution | Administration |
| substance | (mg/kg) | (ml/kg) | method |
| Negative control | — | 10 | po |
| (water) | | | |
| Positive control | 2.5 | 5 | ip |
| (docetaxel) | | | |
| CKD-516 | 4 | 10 | po |
| CKD-516 + | 4 + 2.5 | 10 + 5 | po + ip |
| Docetaxel | | | |

Drug Administration and Identification of Anti-Cancer Activity

An anti-cancer effect of CKD-516 and docetaxel was evaluated by using the prepared animal model. Experimental groups were randomly divided as shown in the table 3 above.
Vehicle: Daily for eight weeks, oral administration;
Docetaxel: 2.5 mg/kg, once a week for eight weeks, intraperitoneal administration;
CKD-516: 4 mg/kg, daily for eight weeks, oral administration; and
CKD-516+Docetaxel: CKD-516 (4 mg/kg, daily for eight weeks, oral administration) to be administered first+Docetaxel (2.5 mg/kg, once a week for eight weeks, intraperitoneal administration).

The anti-cancer activity was determined based on a tumor volume. The tumor volume was obtained by measuring a long axis and a short axis of the tumor by means of electronic callipers (CD-15CPX, Mitutoyo Corp., Japan) and calculating measured values according to a following equation (twice a week):

Tumor volume (mm³)=(Long axis length×Short axis length²)/2.

Statistical Processing

For the tumor volume, statistical processing was performed by using a statistical processing program (GraphPad PRISM® Version 5.0, GraphPad Software, the U.S.).

2. Experimental Results—Anti-Cancer Activity

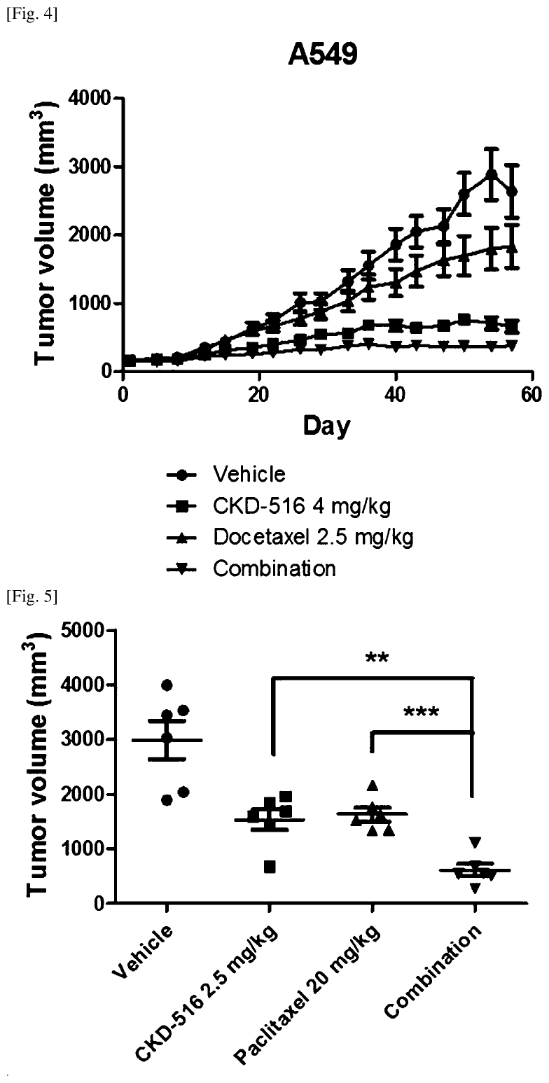

The tumor volume of the negative control group dosed with the excipient only was continuously increased over time. In case of the group dosed with docetaxel alone as a positive control substance, a growth of the tumor was decreased over time compared to the negative control group, but the tumor volume was continuously increased, and there was no significant difference in a growth rate of the tumor compared to the negative control group. In case of the group dosed with CKD-516 alone, the tumor did not almost grow. It was identified for the group dosed with CKD-516 and docetaxel together that the tumor volume is not almost increased compared to an early stage of the experiment, but the tumor volume is remarkably small at the end of the experiment compared to the group dosed with docetaxel alone or CKD-516 alone (FIG. 3). Also, it was identified that the tumor volume is not almost increased, but maintains a certain level for each experiment period, and the tumor volume is remarkably small compared to the group dosed with docetaxel alone or CKD-516 alone during all the experimental periods (FIG. 4).

It suggests that the co-administration of CKD-516 and docetaxel shows an effect of disrupting a growth of cancer tissues to prevent the cancer tissues from growing any more, thanks to a synergy action between a blood vessel-destroying activity by CKD-516 and a cell division-suspending effect by docetaxel.

### <Example 3> Identification of a Synergy Effect Between the Compound of the Formula 2 and Paclitaxel

1. Experimental Method

Preparation for an Animal Model

The four-week old male Balb/c nude mice were purchased from Central Lab Animal Inc.

H460 cancer cell lines, which are human lung cancer cell lines proliferated in vitro, were subcutaneously injected into the mice, such that such cell lines were proliferated in vivo. In 20 to 25 days later, the mice were subjected to euthanasia by means of cervical vertebral dislocation, after which a solid cancer proliferated in the mice was sterilely isolated therefrom to obtain fresh cancer tissues, from which connective tissues, necrotic tissues, skins, etc. were removed.

The cancer tissues were divided by 50 mg in a sterile state, and subcutaneously transplanted into the mice by using a 16-gauge trocar. In 12 days after the transplantation, the mice whose cancer tissues were proliferated up to a certain size, were selected and used for the experiment.

Preparation for an Effective Component

The compound of the formula 2, i.e., CKD-516 was dissolved in 0.9% saline (excipient 1) and prepared in accordance with an administered dose for each group as shown in a following table 4.

Paclitaxel was dissolved in an excipient (excipient 2), which was prepared by mixing cremophor, ethanol and saline at a ratio of 1:1:8, and prepared in accordance with the administered dose for each group as shown in the following table 4.

TABLE 4

| | | Amount of | |
|---|---|---|---|
| | Administered | administered | |
| Administered | dose | solution | Administration |
| substance | (mg/kg) | (ml/kg) | method |
| Negative control | — | 10 | ip |
| (excipient 1) | | | |
| Positive control | 20 | 10 | iv |
| (paclitaxel) | | | |
| CKD-516 | 2.5 | 10 | ip |
| CKD-516 + | 2.5 + 20 | 10 + 10 | ip + iv |
| Paclitaxel | | | |

Drug Administration and Identification of Anti-Cancer Activity

An anti-cancer effect of CKD-516 and paclitaxel was evaluated by using the prepared animal model of tumor. Experimental groups were divided as shown in the table 4 above.
Excipient 1: Once a week for three weeks, intraperitoneal administration;
Paclitaxel: 20 mg/kg, once a week for three weeks, intravenous administration;
CKD-516: 2.5 mg/kg, once a week for three weeks, intraperitoneal administration; and
CKD-516+Paclitaxel: CKD-516 (2.5 mg/kg, once a week for three weeks, intraperitoneal administration) to be administered first+Paclitaxel (20 mg/kg, once a week for three weeks, intravenous administration) to be administered in three days later.

The anti-cancer activity was determined based on a tumor volume. The tumor volume was obtained by measuring a long axis and a short axis of the tumor by means of electronic callipers (CD-15CPX, Mitutoyo Corp., Japan) and calculating measured values according to a following equation (twice a week):

Tumor volume (mm³)=(Long axis length×Short axis length²)/2.

Statistical Processing

All the experimental results were indicated as a mean t standard deviation (Mean t SD), and a comparison was made between each experimental group and the control group by using Student's t-test in order to decide an effect of each experimental group.

2. Experimental Results—Anti-Cancer Activity

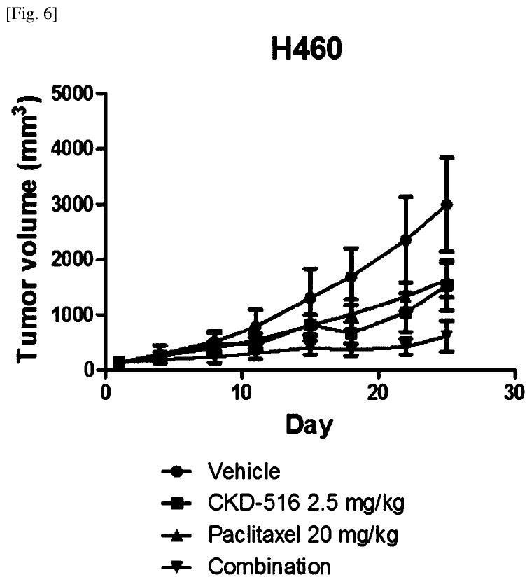

A tumor volume of the negative control group dosed with the excipient only was continuously increased over time. In case of the group dosed with paclitaxel alone as a positive control substance, a growth of the tumor was decreased over time compared to the negative control group, but the tumor volume was continuously increased. Also, the group dosed with CKD-516 alone also showed a similar tendency to the group dosed with paclitaxel. However, it was identified for the group dosed with CKD-516 and paclitaxel together that the tumor volume is not almost increased compared to an early stage of the experiment, but the tumor volume is remarkably small at the end of the experiment compared to the group dosed with paclitaxel alone or CKD-516 alone (FIG. 5). Also, it was identified that the tumor volume is not almost increased, but maintains a certain level during the experimental period (FIG. 6).

It suggests that the co-administration of CKD-516 and paclitaxel shows an effect of disrupting a growth of cancer tissues to prevent the cancer tissues from growing any more, thanks to a synergy action between a blood vessel-destroying activity by CKD-516 and a cell division-suspending effect by paclitaxel.

1. A pharmaceutical composition for preventing or treating cancer, comprising (S)—N-(4-(3-(1H-1,2,4-triazole-1-yl)-4-(3,4,5-trimethoxybenzoyl)phenyl)thiazole-2-yl)-2-amino-3-methylbutanamide represented by a following formula 2 or pharmaceutically acceptable salts thereof; and taxane compound or pharmaceutically acceptable salts thereof.

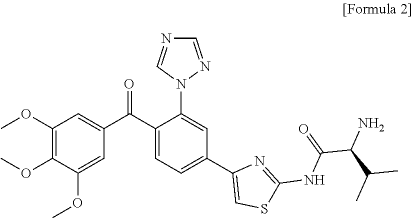 [SMILES: COc1cc(C(=O)c2ccc(-c3csc(NC(=O)[C@@H](N)C(C)C)n3)cc2-n2cncn2)cc(OC)c1OC]

2. The pharmaceutical composition, according to claim 1, wherein a pharmaceutically acceptable salt of (S)—N-(4-(3-(1H-1,2,4-triazole-1-yl)-4-(3,4,5-trimethoxybenzoyl)phenyl)thiazole-2-yl)-2-amino-3-methylbutanamide is hydrochloride.

3. The pharmaceutical composition, according to claim 1, wherein an active metabolite of the compound represented by the formula 2 above is (4-(2-aminothiazole-4-yl)-2-(1H-1,2,4-triazole-1-yl)phenyl)(3,4,5-trimethoxyphenyl)methanone represented by a following formula 3.

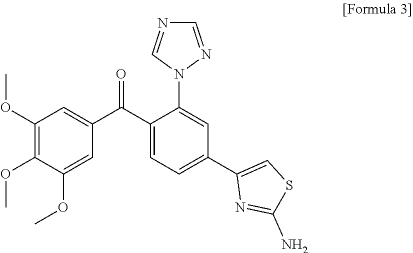 [SMILES: COc1cc(C(=O)c2ccc(-c3csc(N)n3)cc2-n2cncn2)cc(OC)c1OC]

4. The pharmaceutical composition, according to claim 1, wherein the taxane compound is at least one selected from the group comprising paclitaxel, docetaxel, cabazitaxel, larotaxel, ortataxel and tesetaxel.

5. The pharmaceutical composition, according to claim 1, wherein the taxane compound is paclitaxel or docetaxel.

6. The pharmaceutical composition, according to claim 1, wherein the cancer is one selected from colorectal cancer, skin melanoma, lung cancer, gastric cancer, lymphoma and multiple myeloma.

7. The pharmaceutical composition, according to claim 1, wherein the cancer is lung cancer.

8. The pharmaceutical composition, according to claim 1, wherein (S)—N-(4-(3-(1H-1,2,4-triazole-1-yl)-4-(3,4,5-trimethoxybenzoyl)phenyl)thiazole-2-yl)-2-amino-3-methylbutanamide or pharmaceutically acceptable salts thereof are orally or parenterally administered.

9. The pharmaceutical composition, according to claim 1, wherein the taxane compound or pharmaceutically acceptable salts thereof are parenterally administered.

10. The pharmaceutical composition, according to claim 1, wherein (S)—N-(4-(3-(1H-1,2,4-triazole-1-yl)-4-(3,4,5-trimethoxybenzoyl)phenyl)thiazole-2-yl)-2-amino-3-methylbutanamide or pharmaceutically acceptable salts thereof are administered once a day to once every three weeks.

11. The pharmaceutical composition, according to claim 1, wherein the taxane compound or pharmaceutically acceptable salts thereof are administered once a week to once every three weeks.

12. A method for treating a cancer, comprising administering (S)—N-(4-(3-(1H-1,2,4-triazole-1-yl)-4-(3,4,5-trimethoxybenzoyl)phenyl)thiazole-2-yl)-2-amino-3-methylbutanamide represented by a following formula 2 or pharmaceutically acceptable salts thereof; and taxane compound or pharmaceutically acceptable salts thereof into an individual in need thereof.

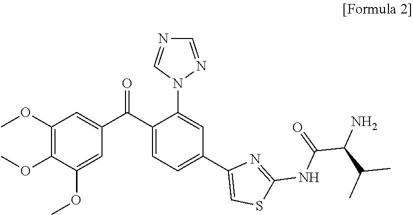 [SMILES: COc1cc(C(=O)c2ccc(-c3csc(NC(=O)[C@@H](N)C(C)C)n3)cc2-n2cncn2)cc(OC)c1OC]

13. Use of (S)—N-(4-(3-(1H-1,2,4-triazole-1-yl)-4-(3,4,5-trimethoxybenzoyl)phenyl)thiazole-2-yl)-2-amino-3-methylbutanamide represented by a following formula 2 or pharmaceutically acceptable salts thereof; and taxane compound or pharmaceutically acceptable salts thereof, in the manufacture of a medicament for treating cancer. 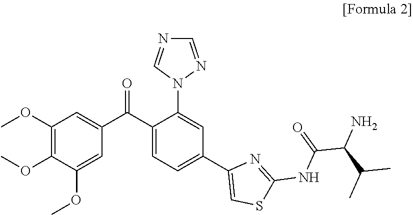 [SMILES: COc1cc(C(=O)c2ccc(-c3csc(NC(=O)[C@@H](N)C(C)C)n3)cc2-n2cncn2)cc(OC)c1OC]
